(19) United States
(12) Patent Application Publication
(10) Pub. No.: US 2025/0352243 A1
(43) Pub. Date: Nov. 20, 2025

(54) APPARATUS AND METHOD FOR CONTROLLING EXTENT OF EXTENSION OF A DEVICE FROM A LUMEN

(71) Applicant: BOSTON SCIENTIFIC SCIMED, INC., Maple Grove, MN (US)

(72) Inventors: Gonzalo Jose SAENZ VILLALOBOS, Alajuela (CR); Fernando VALERIO GUERRERO, Alajuela (CR); Marco Vinicio ARIAS ALPIZAR, Heredia (CR); Valeria ZÚÑIGA VARGAS, Austin, TX (US)

(21) Appl. No.: 19/174,241

(22) Filed: Apr. 9, 2025

Related U.S. Application Data
(60) Provisional application No. 63/647,256, filed on May 14, 2024.

(51) Int. Cl. A61B 17/34; A61B 1/00; A61B 17/00

(52) U.S. Cl. CPC A61B 17/3478; A61B 1/00137; A61B 2017/00358; A61B 2017/00367

(57) ABSTRACT

A device for treating tissue includes a flexible insertion section configured for insertion along a tortuous path to a target site within a body. The section includes a flexible outer sheath extending from a proximal end configured to remain, during use, outside the body accessible to a user of the device; and a cap extending distally from a distal end of the sheath. The cap includes a cap lumen having a proximal portion substantially aligned with and sized and shaped to match a sheath lumen of the sheath. A central portion of the lumen tapers from a maximum dimension at a proximal end open to a distal end of the proximal portion to a reduced dimension at a distal 10 end open to a proximal end of a distal portion of the lumen. An angle of taper of the central portion is between 40 and 20 degrees.

### PRIORITY CLAIM

The present disclosure claims priority to U.S. Provisional Patent Application Ser. No. 63/647,256 filed May 14, 2024; the disclosure of which is incorporated herewith by reference.

### FIELD

The present disclosure relates to an apparatus and a method in which a device is passed through a tube and, depending on the path along which the tube extends, the length of the device within the tube may change. The present disclosure relates to apparatus and method by which a user may control the extent to which such a device may be extended from a distal end of such a tube.

### BACKGROUND

Various endoscopic medical devices are inserted to target sites within the body along tortuous paths. For example, flexible endoscopic devices are often inserted through body lumens to target sites deep within the body. In such cases, where an elongated device extends within a flexible tube from a proximal end outside the body to a distal end adjacent to a target site within the body, the length of the device within the tube may change relative to the tube as the tube is bent along a tortuous path. As understood by those skilled in the art, a greater length of an elongate device will be received within the tube when the tube extends along tortuous path than will be received within the same tube when the tube is extended in a straight configuration. This can be an issue when a user of the device wishes to control or even just to know how far the device extends distally from the distal end of the tube in use. For example, the same operation of an actuator coupled to the device will result in a different length of the device extended from the tube as the path of the tube changes.

### SUMMARY

The present disclosure relates to a device for treating tissue. The device includes a flexible insertion section configured for insertion along a tortuous path to a target site within a living body. The insertion section includes a flexible outer sheath extending from a proximal end configured to remain, during use, outside the body accessible to a user of the device; and a cap extending distally from a distal end of the sheath. The cap includes a cap lumen having a proximal portion substantially aligned with and sized and shaped to match a sheath lumen of the sheath, a central portion of the cap lumen tapering from a maximum dimension at a proximal end open to a distal end of the proximal portion of the cap lumen to a reduced dimension at a distal end open to a proximal end of a distal portion of the cap lumen. An angle of taper of the central portion of the cap lumen is between 40 degrees and 20 degrees.

In an embodiment, the angle of taper of the central portion of the cap lumen is approximately 30 degrees.

In an embodiment, the device further includes a handle coupled to a proximal end of the insertion section, the handle including an actuator configured to move an end effector between a retracted configuration in which the end effector is withdrawn proximally into the sheath and an extended configuration in which the end effector is extended distally out of the cap by a predetermined distance.

In an embodiment, the device further includes a control wire extending from a proximal end coupled to the actuator through the sheath to a distal end coupled to the end effector, operation of the actuator moving the control wire proximally and distally within the sheath to move the end effector between the retracted and extended configurations.

In an embodiment, a diameter of a distal end of the control wire is greater than a diameter of a portion of the end effector that, in the extended configuration extends distally out of the cap.

In an embodiment, contact between the distal end of the control wire and an inner surface of the central portion of the cap determines a distal-most position of the control wire and the end effector, the distal-most position of the control wire being selected to establish a desired distal-most position of the end effector in the extended configuration.

In an embodiment, the end effector includes a needle, wherein the inner surface of the central portion of the cap is configured to guide the needle into the distal portion of the cap lumen.

In an embodiment, the cap has a length of less than 0.51 centimeters.

In an embodiment, the cap has a length no greater than 0.25 centimeters.

In an embodiment, the end effector is a snare and wherein the snare includes a non-expandable proximal portion extending from the distal end of the control wire to a proximal end of an expandable portion configured to expand to an open tissue-receiving configuration when the snare is moved to the extended configuration, a length of the non-expandable proximal portion being selected to establish a desired position of the expandable portion relative to the distal end of the cap when the snare is in the extended configuration.

In an embodiment, the length of the non-expandable proximal portion is between 0.51 centimeters and 0.25 centimeters.

In an embodiment, the cap is formed of a biocompatible metal.

In an embodiment, the cap is formed of stainless steel.

In an embodiment, the cap is formed of aluminum.

In an embodiment, the cap is formed of a biocompatible plastic.

In addition, the present disclosure relates to a method of treating tissue. The method includes inserting into a living body a device including a flexible insertion section configured for insertion along a tortuous path to a target site within the body, wherein the insertion section includes a flexible outer sheath extending from a proximal end configured to remain, during use, outside the body accessible to a user of the device and a cap extending distally from a distal end of the sheath, the cap including a cap lumen having a proximal portion substantially aligned with and sized and shaped to match a sheath lumen of the sheath, a central portion of the cap lumen tapering from a maximum dimension at a proximal end open to a distal end of the proximal portion of the cap lumen to a reduced dimension at a distal end open to a proximal end of a distal portion of the cap lumen, wherein an angle of taper of the central portion of the cap lumen is between 40 degrees and 20 degrees; and operating an actuator of the device to move a control wire proximally and distally within the sheath, the control wire having a distal end coupled to an end effector so that movement of the actuator moves the end effector between a retracted configuration in which the end effector is received within the sheath and an extended configuration in which the end effector is extended distally out of the cap, contact between a distal end of the control wire and an inner surface of the central portion of the cap lumen defining a distal-most position of the end effector relative to the cap when the end effector is in the extended configuration.

In an embodiment, the end effector includes a needle and wherein the central portion of the cap lumen is configured to guide the needle into the distal portion of the cap lumen.

In an embodiment, the cap is formed of a biocompatible metal.

In an embodiment, the end effector includes a snare and wherein the snare includes a non-expandable proximal portion extending from the distal end of the control wire to a proximal end of an expandable portion configured to expand to an open tissue-receiving configuration when the snare is moved to the extended configuration, a length of the non-expandable proximal portion being selected to establish a desired position of the expandable portion relative to the distal end of the cap when the snare is in the extended configuration.

In an embodiment, the cap is formed of a biocompatible plastic.

### BRIEF DESCRIPTION

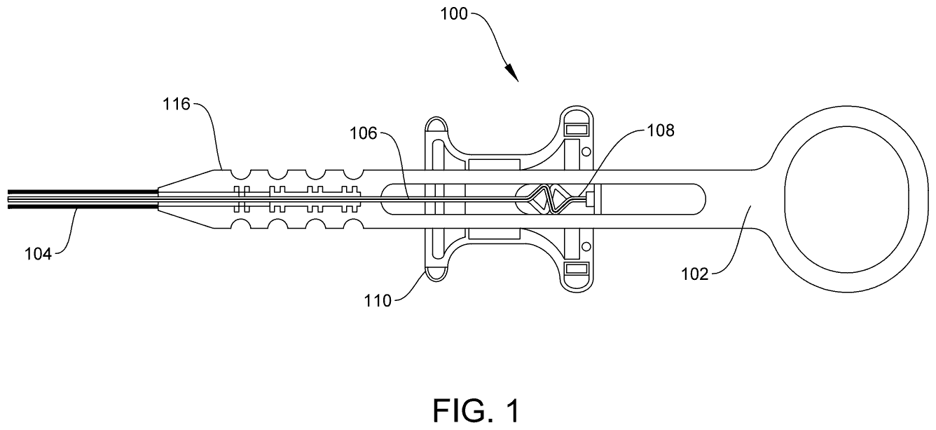

FIG. 1 shows a proximal end of a device according to an exemplary embodiment.

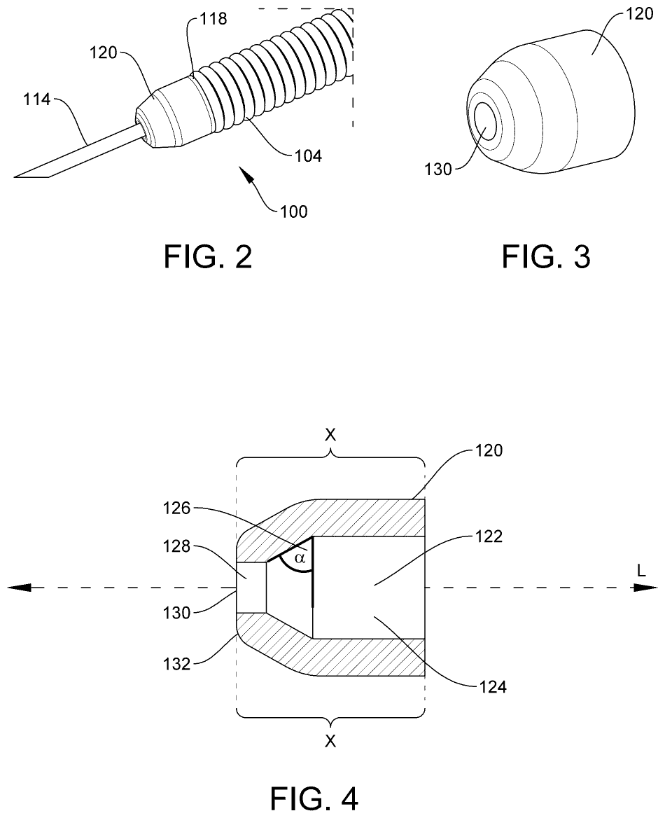

FIG. 2 shows a perspective view of a distal end of the device of FIG. 1.

FIG. 3 shows a perspective view of an end cap of the device of FIG. 1.

FIG. 4 shows a cross-sectional view of the end cap of FIG. 3.

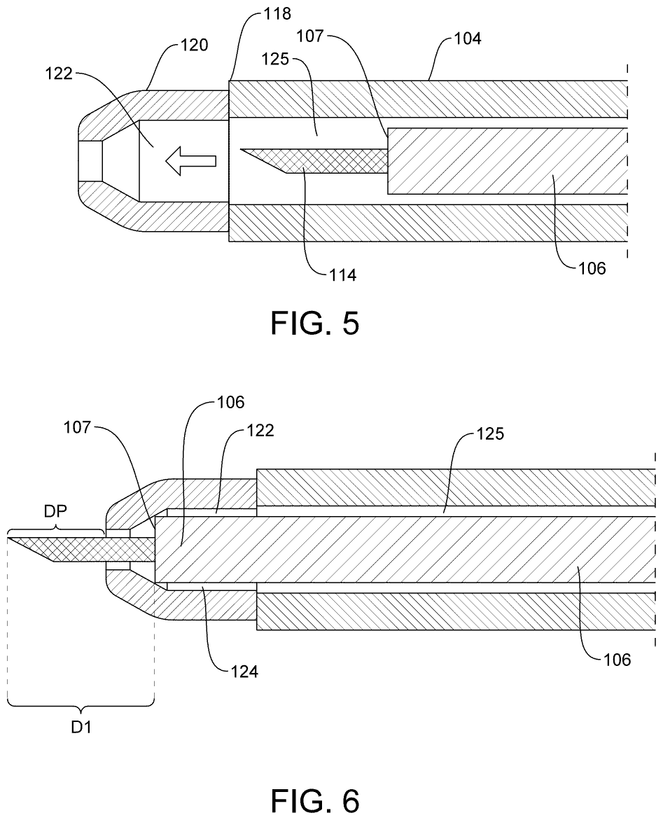

FIG. 5 shows a cross-sectional view of the device of FIG. 2 with a needle in a retracted configuration.

FIG. 6 shows a cross-sectional view of the device of FIG. 2 with the needle in an extended configuration.

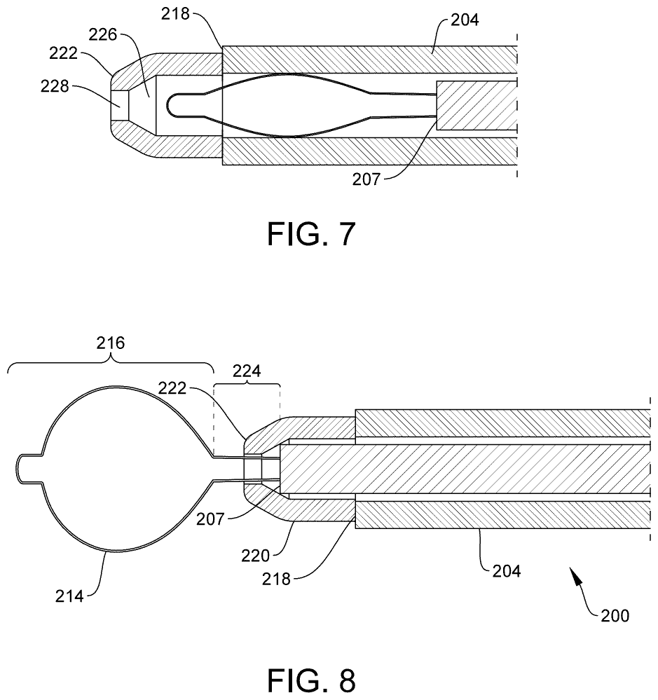

FIG. 7 shows a cross-sectional view of a device of a further embodiment with a snare device in a retracted configuration.

FIG. 8 shows a cross-sectional view of the device of FIG. 7 with the snare device in an extended configuration.

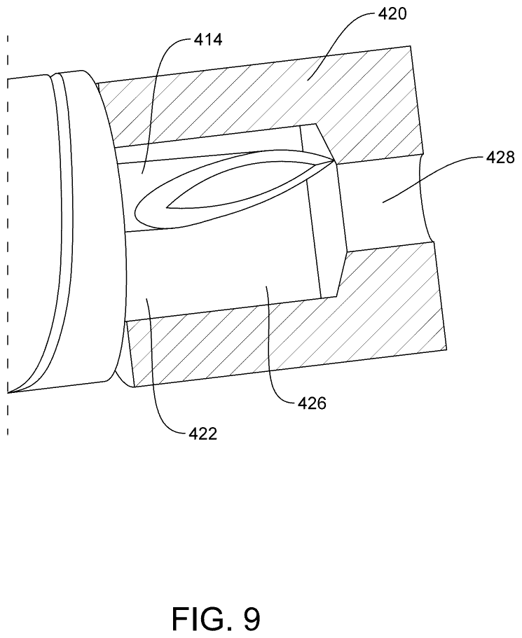

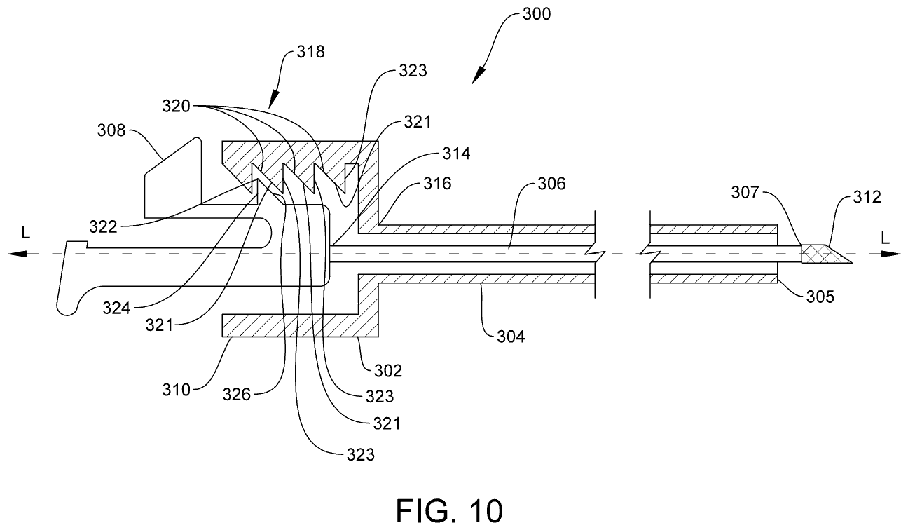

FIG. 9 shows a cross-sectional view of an end cap according to a still further embodiment.

FIG. 10 shows a cross-sectional view of a handle of a device according to an additional embodiment.

### DETAILED DESCRIPTION

The present disclosure may be further understood with reference to the following description and the appended drawings, wherein like elements are referred to with the same reference numerals. The present disclosure relates to endoscopic devices and more particularly to flexible endoscopic devices in which an end effector is coupled to an elongated member so that the end effector can be moved distally and proximally relative to an enclosing sheath so that the end effector is projected (distally) outward from or retracted (proximally) into the sheath. As used in this application, the terms proximal and distal refer to a direction toward (proximal) or away (distal) from a user of the device. Thus, a handle and actuators that remain outside the body accessible to a user form a proximal portion of the device while the distal portion of the device comprises a flexible sheath enclosing an elongate member coupled to the end effector. In use the end distal portion of the device is inserted to a target location in a living body (e.g., through a working channel of an endoscope or other insertion device passing along a natural body passage such as the alimentary canal).

As shown in FIGS. 1-6, a device 100 according to an exemplary embodiment includes a handle 102 with a flexible sheath 104 coupled to a distal end of the handle 102 and extending distally therefrom. In an exemplary embodiment, the sheath 104 is formed as a coil of biocompatible wire (e.g., stainless steel). A control wire 106 extends distally from a proximal end 108 coupled to an actuator 110 through the sheath 104 to a distal end 112 coupled to an end effector. In the embodiment shown in FIGS. 1-6, the end effector is a needle 114 and the control wire 106 is formed as an elongated, flexible shaft coupled at its distal end 107 to the needle 114. Thus, when the actuator 110 is moved proximally and distally relative to a body 116 of the handle 102, the control wire 106 is moved proximally and distally relative to the sheath 104.

In this embodiment, this movement of the actuator 110 relative to the body 116 moves the needle 114 between a retracted position as shown in FIG. 5 and an extended position as shown in FIGS. 2 and 6. FIGS. 2, 4, 5 and 6, show the distal end 118 of the sheath 104 coupled to the proximal end of a cap 120. As seen in FIGS. 5 and 6, the cap 120 includes a lumen 122 that includes a proximal portion 124 that is substantially equal in size to a lumen 125 extending through the sheath 104. In this embodiment, the lumen 125 and the proximal portion 124 of the lumen 122 are generally cylindrical with diameters substantially equal to one another.

However, those skilled in the art will understand that other configurations (e.g., elliptical or other curved shapes) are possible as well. The proximal portion 124 of the lumen 122 opens at its distal end to a tapered portion 126 that necks down from the diameter of the proximal portion 124 to a reduced diameter. The tapered portion 126 opens, in turn, to a distal portion 128 that, in this embodiment, is substantially cylindrical with a diameter substantially equal to the reduced diameter. The distal portion 128 extends distally from the tapered portion 126 to an opening 130 in the distal end 132 of the cap 120.

As can be seen in FIG. 6, the control wire 106 has an outer diameter greater than the reduced diameter of the distal end of the tapered portion 126 and the distal portion 128 of the lumen 122 of the cap 120 while the needle 114 has a diameter selected to permit the needle 114 to be passed through the tapered portion 126 and the distal portion 128 to project distally out of the opening 130. Thus, as the control wire 106 is moved distally via operation of the actuator 110, the needle 114 is moved distally through the sheath 104 and, as the needle 114 enters the tapered portion 126 of the lumen 122, the angled interior surface of the tapered portion 126 guides the needle 114 into the distal portion 128.

This motion brings the distal end 118 of the control wire 106 into contact with the interior surface of the cap 120 within the tapered portion 126 defining a distal-most position to which the control wire 106 can be advanced. Thus, those skilled in the art will understand that, by selecting a distance D1 by which the needle 114 extends distally beyond the distal end 118 of the control wire 106, a projection distance DP by which the needle projects distally out of the cap 120 can be determined. As indicated above, in many applications it is valuable to know accurately how far the end effector (e.g., the needle 114) will extend from the distal end of the device 100 when in the extended position. When the end effector is a needle, the material for the cap 120 is preferably a biocompatible metal such as stainless steel as the hardness of the metal prevents the needle 114 from lodging in the material of the cap 120 as needle 114 is advanced distally into the tapered portion 126. If a softer metal such as aluminum is used for the cap 120, a shallower angle for the tapered portion 126 may be desired as this will reduce the risk that the needle 114 will penetrate into the material of the cap 120. For other end effectors such as snares, biocompatible metals may be used. However, for such end effectors, softer materials such as biocompatible plastics may be used as well.

As indicated in regard to the device 100 of the embodiment of FIGS. 1-6, the angle a at which the tapered portion 126 tapers relative to the proximal portion 124 and the longitudinal axis L is selected so that the needle 114 is guided by the angled surface of the tapered portion 126 toward the distal portion 128. As would be understood by those skilled in the art, if the angle a were made too large (e.g., approaching 90 degrees) the needle 114 may simply press straight into the surface of the tapered portion 126 without being guided toward the axis L so that it may enter the distal portion 128. FIG. 9 shows a situation in which a needle 414 is in contact with a distal interior surface of a cap 420 having a very steeply angled taper in a tapered portion 426 thereof. As can be seen from the figure, the needle 414 is trapped against the distal surface and cannot be advanced into a distal portion 428 of a lumen 422 of the cap 420.

Those skilled in the art will understand that this angle may change depending on the shape and construction of the end effector. For a needle such as the needle 114, the angle a is preferably less than 60 degrees. If the angle a is made too small, the length of the cap 120 would need to be extended to a length that would interfere with the maneuverability of the device 100. That is, if the angle α is made too shallow, the distance required for the diameter of the proximal portion 124 to reduce to the reduced diameter of the distal portion 128 would require a tapered portion having an increased length so that, the cap 120 would interfere with the ability of the device 100 to traverse a tortuous path on the way to the target site. For a cap 120 designed for use in a device with an end effector that is a needle such as the needle 114, the cap may have an angle α between 25 and 35 degrees and more, specifically, the angle α may be 30 degrees.

For example, if the device 100 is to be inserted to a target site within the body via a working channel of an endoscope inserted through a natural body lumen, the endoscope may be required to make one or more tight turns. It is preferable that the device 100 be sufficiently flexible and sized so that the device 100 can pass through the working channel along any path attainable by the endoscope. Thus, the distal portion of the device 100 may for example have an outer diameter of less than 0.20 inches (i.e., 0.508 cm), more preferably, 0.10 inches (i.e., 0.254 cm) or less, and have a flexibility sufficient to turn around a radius of 0.05 inches (i.e., 0.127 cm) or less and at an angle of 90 degrees or less.

To pass through a working channel bent around such a small radius requires that the cap 120 be no longer than 0.20 inches (i.e., 0.508 cm) and more, preferably no longer than 0.10 inches (i.e., 0.254 cm). Thus, for a device 100 having a sheath 104 with an outer diameter of 0.09 inches (i.e., 0.229 cm) and a lumen with a diameter of 0.06 inches (i.e., 0.152 cm) within the sheath 104 and the proximal portion 124, (i.e., having a wall thickness of approximately 0.02 inches (i.e., 0.051 cm)) to maintain the length of the cap 120 below 0.01 inches (i.e., 0.025 cm), the angle α must be 35 degrees or less and the opening 130 of the cap 120 may have an outer diameter of 0.03 inches (i.e., 0.076 cm). Those skilled in the art will understand that these dimensions may be varied in any desired manner so long as the device remains capable of passing through the insertion device to the target site within the body and so long as the inner surface of the tapered portion of the cap is sufficiently angled to guide the end effector to the distal portion of the lumen of the cap. The angle α may have a tolerance in degrees (e.g., plus or minus 5 degrees) and the lengths, including the radii and diameters, may have a tolerance in length (e.g., plus or minus 0.05 inches (i.e., 0.127 cm)).

FIGS. 7 and 8 show a device 200 which is constructed substantially similarly to the device 100 except that the end effector of the device 200 is a snare. Specifically, the device 200 includes a handle (not shown) the same as the handle 102 of the device 100. The device 200 includes a sheath 204 extending distally from the handle to a distal end 218. A control wire 206 extends within the sheath from an actuator (not shown) of the handle to a distal end 207 coupled to a snare 214. As shown in FIG. 7, when in a retracted position, the snare 214 is held entirely within the sheath 204 and the cap 220. In the extended position as shown in FIG. 8, the snare 214 is fully extended so that an expanded portion 216 of the snare 214 (biased to assume a circular shape when no longer constrained by the sheath 204) is projected distally from the cap 220 ready to receive tissue.

As would be understood by those skilled in the art, it is preferred that the expanded portion 216 of the snare 214 will be located a desired distance from the distal end 222 of the cap 220 when in the extended position. To achieve this, a length of a non-expanded portion 224 of the snare 214 is selected so that, when the distal end 218 of the sheath 204 reaches a distal-most position (i.e., when contact between the inner surface of the tapered portion 226 of a lumen of the cap 220 and sheath 204 prevents further distal movement of the sheath 204), the snare 214 is positioned relative to the distal end 222 of the cap 220 as desired.

In the case of the device 200 including the snare 214, the length of the non-expanded portion 224 is preferably between 0.10 inches (i.e., 0.254 cm) and 0.20 inches (i.e., 0.508 cm). In this example, the sheath 204 contacts the inner surface of the tapered portion 226 in the distal-most position at a location separated from the distal end 222 of the cap 220 by between 0.04 and 0.02 inches (i.e., 0.051 cm) and more preferably by 0.03 inches (i.e., 0.076 cm). Thus, the expanded portion 216 of the snare 214 extends from a proximal end which, in the extended position is approximately 0.03 inches (i.e., 0.076 cm) from the distal end 222 of the cap 220. Those skilled in the art will understand that these dimensions may be varied in any desired manner so long as the device remains capable of passing through the insertion device to the target site within the body and so long as the inner surface of the tapered portion of the cap is sufficiently angled to guide the end effector to the distal portion of the lumen of the cap. For example, the angle may have a tolerance in degrees (e.g., plus or minus 5 degrees) and the lengths, including the radii and diameters, may have a tolerance in length (e.g., plus or minus 0.05 inches (i.e., 0.127 cm)).

In addition, those skilled in the art will understand that the outer surface of the cap 120, 220 is preferably formed in a tapered atraumatic shape. This atraumatic shape may generally follow the contour of the tapered portion 126, 226 except that the slope of the outer surface may, in an exemplary embodiment follow a taper that begins at a location T along the longitudinal axis L aligned with the proximal end of the tapered portion 126, 226 and this outer surface may taper along the combined lengths of the tapered portions 126, 226 and the distal portions 128, 228.

In this exemplary embodiment a distal surface of the cap is substantially perpendicular to the longitudinal axis L. Thus, if the difference between the diameter of the proximal portion of the cap and the distal end of the cap is similar to the difference between the diameter of the proximal portion of the lumen of the cap and the distal portion of the lumen of the cap, the taper of the outer surface of the cap may be slightly more gradual than the taper of the tapered portion of the lumen of the cap. However, as would be understood by those skilled in the art, the shape of the atraumatic tip may be varied in any desired direction so long as the structural integrity of the cap is not affected.

FIG. 10, shows a handle 302 of a device 300 according to an additional embodiment in which the handle 302 includes a mechanism for permitting a control wire 306 to be extended in length by an amount selected to compensate for the change in the relative position of the distal end 307 of the control wire 306 when a sheath 304 within which the control wire 306 is received is bent along a tortuous path (e.g., when the sheath 304 is inserted to a target site within the body along a path including multiple bends). The distal end 307 of the control wire 306 is shown in this example coupled to the proximal end of a needle 312 although, as would be understood by those skilled in the art, this mechanism for permitting the control wire 306 to be extended in length by a user-selected amount relative to the distal end 305 of the sheath 304, may be constructed in the same manner regardless of the type of end effector coupled to the control wire 306.

The handle 302 includes an actuator 308 slidably mounted relative to a body 310 of the handle 302. The proximal end 314 of the control wire 306 is coupled to the actuator 308 while a proximal end 316 of the sheath 304 is coupled to the body 310 of the handle 302. A ratchet mechanism 318 is formed between the actuator 308 and the body 310 of the handle 302 to selectively lock the actuator 308 in a desired position relative to the body 310. The ratchet mechanism 318 of this embodiment includes a series of ramped projections 320 sized, shaped, and positioned to mate with a corresponding ramped projection 322 formed on the actuator 308.

Specifically, each of the projections 320 has a ramped proximal surface 321 and a distal surface 323 that extends transverse to the direction of travel of the actuator 308 as it is slid relative to the body 310 of the handle 302. In this embodiment, as the actuator 308 is configured to move proximally and distally relative to the body 310 along a longitudinal axis L of the handle 302, the distal surface 323 of the projections 320 extend generally perpendicularly to the axis L. The projection 322 of this embodiment includes a proximal surface that extends transverse to the axis L and a ramped distal surface 326. As would be understood by those skilled in the art, when a user wants to extend the control wire 306 distally (e.g., to compensate for withdrawal of the distal end 307 of the control wire 306 relative to the distal end 305 of the sheath 304), the user pushes the actuator 308 distally relative to the body 310 of the handle 302 so that the projection 322 slides distally over the proximal-most projection 320 until the proximal end of the distal surface 326 reaches the distal end of the proximal surface 321 of this proximal-most projection 320.

At this point, the projection 322 slides radially outward away from the axis L and the transverse distal surface 323 of the projection 320 mates with the transverse proximal surface 324 of the projection 322 preventing (temporarily) proximal movement of the actuator 308 relative to the body 310. Thus, the control wire 306 is moved by a corresponding distance into the sheath 304 and the position of the distal end 307 of the control wire 306 (and, consequently, the position of the needle 312) relative to the distal end 305 of the sheath 304 can be adjusted by the user. If this distal movement of the actuator 308 does not achieve the desired position of the needle 312 (e.g., as confirmed via imaging, etc.) the user may advance the actuator 308 to the next location (defined by the next most proximal projection 320) and repeat until the desired location is achieved. If the user wishes to withdraw the needle 312 proximally, the user simply depresses the actuator 308 toward the axis L to move the projection 322 out of engagement with the projection 320 and pulls the actuator 308 proximally by the desired amount.

Thus, the user can make any necessary compensations for changes in position of the needle 312 relative to the distal end 305 of the sheath 304 and the needle 312 will remain locked in this position until the actuator 308 is further adjusted by the user. Those skilled in the art will understand that this ratchet mechanism can be combined with the sliding actuator 110 or a similar mechanism so that the actuator 308 simply establishes an initial position of the needle 312 (or other end effector) while the sliding actuator 110 is used to move the needle 312 between the retracted and extended configurations as described above.

It may be noted by those knowledgeable in the art that any of the above embodiments may be combined in any manner not inconsistent with their operation and design to provide a system to enable ostomy management utilizing any or all of the characteristics of the various embodiments.

1-15. (canceled)

16. A device for treating tissue, comprising:
a flexible insertion section configured for insertion along a tortuous path to a target site within a living body, the insertion section including:
a flexible outer sheath extending from a proximal end configured to remain, during use, outside the body accessible to a user of the device; and
a cap extending distally from a distal end of the sheath, the cap including a cap lumen having a proximal portion substantially aligned with and sized and shaped to match a sheath lumen of the sheath, a central portion of the cap lumen tapering from a maximum dimension at a proximal end open to a distal end of the proximal portion of the cap lumen to a reduced dimension at a distal end open to a proximal end of a distal portion of the cap lumen, wherein an angle of taper of the central portion of the cap lumen is between 40 degrees and 20 degrees.

17. The device of claim 16, wherein the angle of taper of the central portion of the cap lumen is approximately 30 degrees.

18. The device of claim 16, further comprising:
a handle coupled to a proximal end of the insertion section, the handle including an actuator configured to move an end effector between a retracted configuration in which the end effector is withdrawn proximally into the sheath and an extended configuration in which the end effector is extended distally out of the cap by a predetermined distance.

19. The device of claim 18, further comprising:
a control wire extending from a proximal end coupled to the actuator through the sheath to a distal end coupled to the end effector, operation of the actuator moving the control wire proximally and distally within the sheath to move the end effector between the retracted and extended configurations.

20. The device of claim 19, wherein a diameter of a distal end of the control wire is greater than a diameter of a portion of the end effector that, in the extended configuration extends distally out of the cap.

21. The device of claim 20, wherein contact between the distal end of the control wire and an inner surface of the central portion of the cap determines a distal-most position of the control wire and the end effector, the distal-most position of the control wire being selected to establish a desired distal-most position of the end effector in the extended configuration.

22. The device of claim 21, wherein the end effector includes a needle, wherein the inner surface of the central portion of the cap is configured to guide the needle into the distal portion of the cap lumen.

23. The device of claim 16, wherein the cap has a length of less than 0.51 centimeters.

24. The device of claim 16, wherein the cap has a length no greater than 0.51 centimeters.

25. The device of claim 21, wherein the end effector is a snare and wherein the snare includes a non-expandable proximal portion extending from the distal end of the control wire to a proximal end of an expandable portion configured to expand to an open tissue-receiving configuration when the snare is moved to the extended configuration, a length of the non-expandable proximal portion being selected to establish a desired position of the expandable portion relative to the distal end of the cap when the snare is in the extended configuration.

26. The device of claim 25, wherein the length of the non-expandable proximal portion is between 0.51 centimeters and 0.25 centimeters.

27. The device of claim 23, wherein the cap is formed of a biocompatible metal.

28. The device of claim 27, wherein the cap is formed of stainless steel.

29. The device of claim 27, wherein the cap is formed of aluminum.

30. The device of claim 25, wherein the cap is formed of a biocompatible plastic.

31. A method of treating tissue, comprising:
inserting into a living body a device including a flexible insertion section configured for insertion along a tortuous path to a target site within the body, wherein the insertion section includes a flexible outer sheath extending from a proximal end configured to remain, during use, outside the body accessible to a user of the device and a cap extending distally from a distal end of the sheath, the cap including a cap lumen having a proximal portion substantially aligned with and sized and shaped to match a sheath lumen of the sheath, a central portion of the cap lumen tapering from a maximum dimension at a proximal end open to a distal end of the proximal portion of the cap lumen to a reduced dimension at a distal end open to a proximal end of a distal portion of the cap lumen, wherein an angle of taper of the central portion of the cap lumen is between 40 degrees and 20 degrees; and
operating an actuator of the device to move a control wire proximally and distally within the sheath, the control wire having a distal end coupled to an end effector so that movement of the actuator moves the end effector between a retracted configuration in which the end effector is received within the sheath and an extended configuration in which the end effector is extended distally out of the cap, contact between a distal end of the control wire and an inner surface of the central portion of the cap lumen defining a distal-most position of the end effector relative to the cap when the end effector is in the extended configuration.

32. The method of claim 31, wherein the end effector includes a needle and wherein the central portion of the cap lumen is configured to guide the needle into the distal portion of the cap lumen.

33. The method of claim 32, wherein the cap is formed of a biocompatible metal.

34. The method of claim 31, wherein the end effector includes a snare and wherein the snare includes a non-expandable proximal portion extending from the distal end of the control wire to a proximal end of an expandable portion configured to expand to an open tissue-receiving configuration when the snare is moved to the extended configuration, a length of the non-expandable proximal portion being selected to establish a desired position of the expandable portion relative to the distal end of the cap when the snare is in the extended configuration.

35. The method of claim 34, wherein the cap is formed of a biocompatible plastic.
